(19) United States
(12) Patent Application Publication
(10) Pub. No.: US 2022/0392639 A1
(43) Pub. Date: Dec. 8, 2022

(54) Using Machine Learning-Based Trait Predictions For Genetic Association Discovery

(71) Applicant: GOOGLE LLC, Mountain View, CA (US)

(72) Inventors: Cory McLean, Mountain View, CA (US); Babak Alipanahi, Mountain View, CA (US); Justin Cosentino, Mountain View, CA (US); Sonia Phene, Mountain View, CA (US); Andrew Carroll, Mountain View, CA (US)

(21) Appl. No.: 17/770,174

(22) PCT Filed: Oct. 13, 2020
(86) PCT No.: PCT/US2020/055348

§ 371 Date: Apr. 19, 2022

Related U.S. Application Data
(60) Provisional application No. 62/928,555, filed on Oct. 31, 2019.

(51) Int. Cl. G16H 50/20; G16H 50/70; G16B 20/00; G06N 3/04

(52) U.S. Cl. CPC G16H 50/20; G16H 50/70; G16B 20/00; G06N 3/0454

(57) ABSTRACT

A method for producing highly accurate, low cost phenotype labels for a cohort of individual using a machine learning model. The model is trained to predict phenotype labels from routine clinical data. We describe routine clinical data in the form of fundus images and making predictions as to phenotypes associated with eye diseases, such as glaucoma, however the methodology is more generally applicable to phenotype assignment from clinical data. The model is applied to a cohort of interest which includes both genomic data and the same type of routine clinical data. The model produces phenotype labels for each of the members of the cohort of interest. We then conduct a genetic association test (e.g., GWAS) on the cohort of interest using the phenotype labels produced by the model along with associated genomic data and identify genomic information (e.g., specific loci in the genome) associated with the phenotype.

### BACKGROUND

The term “phenotype” refers to the set of observable characteristics of an individual resulting from the interaction of its genotype with the environment. The term “phenotyping” refers to a methodology of assigning a particular label to such characteristics for a particular individual.

Currently, the task of phenotyping occurs on a spectrum in which high accuracy of a phenotype assignment requires an associated high cost to acquire, or lower accuracy can be achieved at a lower cost. The task of accurately phenotyping large cohorts (e.g., a collection of clinical data for thousands or tens of thousands of individuals) is a substantial challenge. Acquiring clinical phenotypes can be costly, time-consuming, or infeasible. Examples of the high-accuracy, high-cost phenotypes are phenotypes derived in clinical settings or as part of an explicit research program focused on a disease of interest. Each of these methods requires interaction with individuals in the cohort to determine additional phenotypes for which genetic links can be analyzed.

By contrast, self-reported phenotypes can be easier to obtain but are often less accurate or susceptible to multiple forms of bias. In particular, low cost self-reported phenotypes are subject to ascertainment bias in the population of people who participate in the program, as well as self-selection and non-response biases. Low-accuracy, low-cost phenotypes can be gathered through self-reporting, e.g., from web-based questionnaires such as found on websites such as 23andMe.com.

Discovering the influence of genetic variation on phenotypes (i.e. traits or disease susceptibility) requires collecting a cohort of individuals with both genetic information and accurate phenotype labels. This tradeoff of accuracy and cost in generating phenotype labels poses a challenge to discovering the genetic contributions to disease. Many common diseases have been shown to have hundreds or thousands of genetic variants each with a very small contribution to overall disease risk. Both sample size and phenotype accuracy are required to maximize statistical power to discover genetic variant links to phenotypes.

This disclosure relates to a method for accurately generating phenotype labels for a large cohort of interest, and the subsequent use of the labeled cohort along with associated genomic data for genetic association discovery. The method overcomes the hurdles described above in accurately assigning phenotype labels to large cohorts, namely cost, time-consuming effort and infeasibility, while also avoiding the various biases and lack of accuracy in self-reporting phenotypes.

### SUMMARY

A method is disclosed for identifying an association between genomic information and a phenotype associated with a particular disease or medical condition. The method includes a step of training a machine learning model to predict phenotype status from a training dataset in the form of phenotype-labeled routine clinical data for a multitude of individuals. This labeling can be a mixture of manual labeling or automatic labeling with manual review/adjudication, and can be applied to both training data generated in real-world settings and synthetically-generated training data.

Next, the model is applied to a cohort of interest that contains both genomic data and the same routine clinical data (e.g., fundus images) used as input to the model during training. The model produces phonotype labels for the members of the cohort of interest. The method continues with a step of conducting a genetic association test on the cohort of interest using the phenotype labels produced in in the previous step along with associated genomic data. Such a study identifies genomic information associated with the phenotype. One method for associating genetic variants with a phenotype is a genome-wide association study (GWAS), which is described at some length below.

The inventors describe an application of their methodology in which the phenotype labels are associated with glaucoma. The training dataset consisted of 80,232 fundus images from individuals not in the UK Biobank (UKB). Phenotype labels for this training dataset were adjudicated by a team of ophthalmologists, optometrists, and glaucoma specialists. This data formed the majority of training images previously used to train a model of referable GON risk and multiple optic nerve head features that performed on par with glaucoma specialists in three validation datasets, described in a paper (S. Phene et al., Deep Learning for Glaucoma Specialists, American Academy of Ophthalmology, published online Jul. 24, 2019). The inventors trained an ensemble of ten deep convolutional networks using the 80,232 fundus images and used the model to predict glaucomatous optic neuropathy (GON), vertical cup-to-disk ratio (VCDR), retinal nerve fiber layer defect, disc hemorrhage, and focal notching presence phenotypes.

They then applied this trained model to a cohort of fundus images from 80,271 glaucoma patients who were in the UK Biobank, and assigned a phenotype label of predicted GON risk to each member of this cohort. The phenotype prediction was a continuous variable, not a binary label. Genomic data was present for every individual in this cohort. A GWAS study was then conduct for this cohort. The inventors discovered 22 genome-wide significant loci (i.e., specific locations in the genome, each identified with a reference single nucleotide polymorphism (SNP) ID number, or “rs” ID number) associated with the GON risk phenotypes in individuals of European ancestry. Fourteen of such loci replicate known genomic associations with primary open angle glaucoma (POAG) or endophenotypes like intraocular pressure and VCDR. The remaining 8 loci are novel or have equivocal prior evidence for glaucoma association. A description of these loci is set forth later in this document. While we try to map each locus (a region of the genome) to the likely gene that it influences, such a mapping is an estimate based solely on genome location. However, there are well-known examples of specific genomic regions influencing genes much further away, and so the loci are not necessarily associated firmly with specific genes.

While the application will provide as an example the phenotype labeling of a cohort based on fundus images as the clinical data, in theory the same methodology can be used with other types of clinical data. For example, alternative embodiments of this disclosure are contemplated extending the prediction capacity for other phenotypes from color fundus images, including phenotypes associated with diabetic retinopathy and macular degeneration. Additionally, the methods are applicable to other routine clinical data types including but not limited to electronic health records, medical imaging data, and laboratory test values. In these latter situations, the trained machine learning model for generating phenotype predictions may vary, and may for example take the form of long-short term memory models, transformer models, convolutional neural networks and fully-connected neural networks. For example, the models described in Google Published PCT application of Kai Chen et al., publication no. WO 2019/022779 (describing several different model architectures for making future health predictions from electronic health records) could be used.

### BRIEF DESCRIPTION OF THE DRAWINGS

FIGS. 1A and 1B are a diagram of a method or workflow for highly accurate low-cost phenotyping and associated genomic association studies of this disclosure.

FIG. 1A shows the workflow for a one-time model training procedure. A training dataset (possibly smaller and/or unrelated to the cohort of interest with both genomics and clinical data) has extensive curation of phenotype labels to determine individual phenotype status, and is used to train a model to predict the phenotype.

FIG. 1B illustrates the workflow of the trained model from FIG. 1A to a cohort of interest to generate phenotype values and their subsequent use in a genomic association study for genetic discovery.

### DETAILED DESCRIPTION

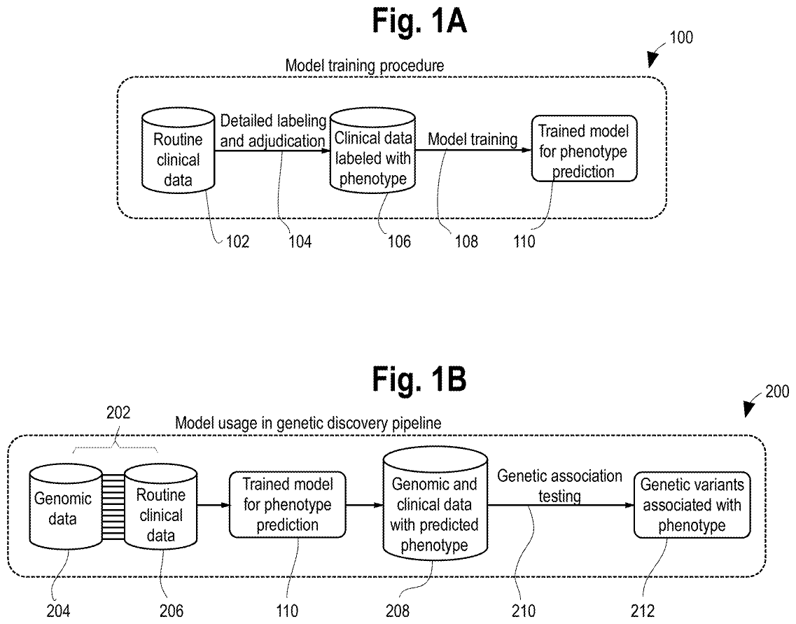

A method is described for identifying an association between genomic information and a phenotype associated with a particular disease or medical condition. The methodology or workflow is shown in FIGS. 1A and 1B and consists of two parts, namely a first part 100 (model training procedure, FIG. 1A) and a second part 200 (FIG. 1B), in which the model trained in the first part 100 is used to label a cohort of interest and subsequent genetic association testing is performed to produce a list of genetic variants associated with one or more phenotypes.

Referring now in particular to FIG. 1A, this figure shows a model training exercise. A training dataset 102 includes routine clinical data, such as electronic medical records, image data (e.g., retinal images, etc.). This training dataset 102 is subject to detailed phenotype labeling and adjudication, typically by human experts, to assign phenotype labels to the individuals in the training dataset. The result of this phenotyping process 104 is a phenotype labeled training dataset 106 of routine clinical data associated with particular phenotype labels. This dataset 106 is then subject to a machine learning model training exercise as indicated at step 108. This model training exercise could take a variety of forms, including training a neural network, training a deep convolutional neural network, ensemble of deep convolutional neural networks, etc. which learns to associate phenotype labels with particular data clinical data such that it can accurately classify or label new instances of routine clinical data (of the same type as in the training dataset 102) with a phenotype label. Examples of this model training process 108 will be given below.

The result of the model training exercise 108 is a trained model 110 for phenotype prediction from clinical data. An example of the trained model for training eye-related clinical data to produce phenotype labels associated with glaucoma risk is described in detail on the paper of S. Phene et al., Deep Learning for Glaucoma Specialists, American Academy of Ophthalmology, published online Jul. 24, 2019. The methodology of this paper, including the machine learning architecture, can be extended to other types of clinical datasets. For example, the method of process 100 can be applied to alternative, routine data including but not limited to electronic health records, medical imaging data, and laboratory test values. In these latter situations, the trained machine learning model 110 generating phenotype predictions may vary, and may for example take the form of long-short term memory models, transformer models, convolutional neural networks and fully-connected networks. For example, the models described in Google Published PCT application of Kai Chen et al., publication no. WO 2019/022779 (describing several different model architectures for making future health predictions from electronic health records) could be used. The entire content of the WO 2019/022779 patent application publication is incorporated by reference herein. See also Juan Banda et al., Advances in Electronic Phenotyping: From Rule-Based Definitions to Machine Learning Models, Annual Review of Biomedical Data Science, vol. 1, pp. 53-68 (July 2018), the content of which is incorporated by reference herein.

Referring now to FIG. 1B, a workflow 200 is shown in which trained model 110 from FIG. 1A is applied to a cohort of interest to generate phenotype values and their subsequent use (in step 210) in a genomic association study for genetic discovery resulting in a list 212 of genetic variants which are associated with a particular phenotype. Workflow 200 includes two parts. Data for a cohort of interest 202 including both genomic data 204 and clinical data 206 (of the same type of routine clinical data 102 used for model training in workflow 100 of FIG. 1A) is obtained. Data for the cohort of interest could be obtained from publicly-available sources, such as for example the UK Biobank. The genomic data 204 could take the form of full genomic sequencing or sequencing of particular genes or genomic regions. The clinical data could consist of demographic data, test values, image data, medical record data, etc. This cohort of interest 202 is initially unlabeled as to the phenotypes of interest; the procedure of FIG. 1B assigns accurate phenotype labels to the cohort 202, automatically, and without requiring any substantial human effort, as would be required by prior art methods discussed previously.

In particular, in FIG. 1B, the trained model 110 from FIG. 1A is applied to this cohort of interest 202 whereby the model 110 produces phenotype labels for each of the members of the cohort of interest 202 from the routine clinical data. Moreover, because the routine clinical data 206 is associated with genomic data, the result of the application of the trained model 110 to the cohort 202 is a dataset (208) of phenotype-labeled clinical data which is also associated with genomic data. In order to discover particular genetic variants which are associated with the phenotype labels, a genetic association test 210 is conducted on the dataset 208. This genomic association test is designed to identify particular genomic information (e.g., genetic loci, single nucleotide polymorphisms, etc.) which are associated or linked to the phenotype labels. While any of the known genetic association tests for making such discoveries could be used, in this disclosure we particularly contemplate the use of a genome-wide association study (GWAS) for the procedure 210. This procedure results in a list of genetic variants that are associated with phenotypes.

A genome-wide association study (GWAS) is an experimental design used to detect associations between genetic variants and traits (phenotypes) in samples from populations. The primary goal of these studies is to better understand the biology of disease, under the assumption that a better understanding will lead to prevention or better treatment. A good overview of GWAS methods is set forth in the educational article of William S. Bush et al., Chapter II Genome-Wide Association Studies, PLOS Computational Biology, December 2012, Volume 8, Issue 12, the content of which is incorporated by reference herein.

The path from GWAS to biology is not straightforward because an association between a genetic variant at a genomic locus and a trait is not directly informative with respect to the target gene or the mechanism whereby the variant is associated with phenotypic differences. However, as described in the review article of Peter M. Visscher et al., 10 Years of GWAS Discovery: Biology, Function, and Translation, The American Journal of Human Genetics vol. 101, pp. 5-22 (Jul. 6, 2017), new types of data, new molecular technologies, and new analytical methods have provided opportunities to bridge the knowledge gap from sequence to consequence. The content of the Visscher et al. reference, including the descriptions of the analysis methods of Table 1 of the Visscher et al. cited in the article, is also incorporated by reference herein. GWASs have also been successfully implemented for better defining the relative role of genes and the environment in disease risk, assisting in risk prediction, and investigating natural selection and population differences.

### Example

An example of the use of the methodology of FIGS. 1A and 1B will now be set forth. The model 110 of FIG. 1A was trained to generate a phenotype label of referable glaucomatous optic neuropathy (GON) using retinal fundus color photographic images as the routine clinical data (102) and using such labels in FIG. 1B in a cohort of interest to discover genetic influences on primary open angle glaucoma (POAG) using GWAS.

In FIG. 1A, the training dataset 102 consisted of 80,232 fundus images from individuals not in the UK Biobank (UKB) adjudicated by a team of ophthalmologists, optometrists, and glaucoma specialists in step 104. This data formed the majority of training images previously used to train a model of referable GON risk and multiple optic nerve head features that performed on par with glaucoma specialists in three validation datasets, see the S. Phene et al. article cited previously for details.

In the model training process 100, we trained a model 110 in the form of an ensemble of ten deep convolutional networks using the 80,232 fundus images. This model 110 is preferably designed such that the phonotype label produced by the model in the form of a continuous variable probability prediction. For example, the phenotype label can be an ensemble average from the ten deep convolutional neural networks and expressed as a probability of a given phenotype label being correct of between 0 and 1.

In FIG. 1B, the model 110 is used to predict GON, vertical cup-to-disk ratio (VCDR), retinal nerve fiber layer defect, disc hemorrhage, and focal notching presence phenotypes for all 80,271 individuals in the UKB with fundus images. GON prediction performance was validated in the subset of UKB images that had undergone adjudication previously (N=378; AUC=0.902, AUPRC=0.579).

At step 210, we performed a genome-wide association study on the predicted GON risk phenotype in the UKB individuals of European ancestry (N=58,503). Of 22 genome-wide significant loci, see Table 1 below, 14 loci replicate known associations with POAG or endophenotypes like intraocular pressure and VCDR. The remaining 8 are novel or have equivocal prior evidence for glaucoma association. The loci are identified with an rslD number identifier, as is common in the art.

TABLE 1

| | |
|---|---|
| rs12024620 | (p = 4.55 × 10\hat{ }−08) |
| rs4658101 | (p = 4.81 × 10\hat{ }−23) |
| rs1346789 | (p = 2.34 × 10\hat{ }−11) |
| rs4858683 | (p = 2.88 × 10\hat{ }−11) |
| rs34025447 | (p = 8.19 × 10\hat{ }−09) |
| rs2448966 | (p = 2.70 × 10\hat{ }−10) |
| rs562380403 | (p = 6.80 × 10\hat{ }−09) |
| rs72655753 | (p = 8.74 × 10\hat{ }−10) |
| rs1360589 | (p = 3.71 × 10\hat{ }−46) |
| rs11244049 | (p = 2.13 × 10\hat{ }−08) |
| rs7916697 | (p = 3.17 × 10\hat{ }−26) |
| rs1223102 | (p = 6.07 × 10\hat{ }−11) |
| rs7936928 | (p = 1.83 × 10\hat{ }−09) |
| rs11115955 | (p = 2.88 × 10\hat{ }−30) |
| rs4899012 | (p = 2.39 × 10\hat{ }−15) |
| rs74056339 | (p = 2.23 × 10\hat{ }−08) |
| rs8053277 | (p = 2.92 × 10\hat{ }−11) |
| rs123698 | (p = 5.73 × 10\hat{ }−12) |
| rs928203 | (p = 4.31 × 10\hat{ }−10) |
| rs545472419 | (p = 4.86 × 10\hat{ }−08) |
| rs5752776 | (p = 4.15 × 10\hat{ }−27) |
| rs34611740 | (p = 5.19 × 10\hat{ }−10) |

Our method for conducting GWAS on this dataset is set forth below. It will be understood by persons skilled in the art that the following is a representative but not limiting example of how GWAS can be conducted. Further examples are set forth in the two GWAS papers cited previously, as well as in many references in the scientific literature, including the list of papers cited in the article of Peter M. Visscher et al., 10 Years of GWAS Discovery: Biology, Function, and Translation, The American Journal of Human Genetics vol. 101, pp. 5-22 (Jul. 6, 2017). Accordingly the following description is offered by way of example only.

a) Shard UKB Imputed Genotype Data and Convert to PLINK Format

Note: This is an implementation detail to make the process run faster by using multiple computers. It is not core to the idea of running GWAS, but is included here for the sake of completeness. Imputed genotype data contains, for each variant to be tested for association with the trait of interest, an estimate of the number of alternate alleles each individual in the cohort contains. Since humans are diploid organisms, this estimate is a number between 0 and 2 (possibly fractional to represent uncertainty in the estimate). Sharding the imputed data involves splitting a single file containing all imputed data into multiple disjoint files, each containing data for a subset of all variants.

b) Perform GWAS on all Selected Phenotypes and Settings (e.g. Adding Intraocular Pressure (IOP) as a Covariate to Discover Non-IOP Related Genetic Factors)

As discussed in the links above, in a GWAS, each variant is tested independently for significance of association with the trait of interest. This is typically done by fitting a null model in which the trait outcome y is a function of non-variant covariates (e.g. age, sex, body mass index (bmi), and 5-20 principal components of genetic ancestry) and comparing the model fit to one in which the estimated number of non-reference alleles of the variant of interest is also included in the model.

c) Perform QC on GWAS Results (QQ-Plots, Genomic Correction, Variant QC)

Quality control (QC) measures are crucial to ensure the validity of the GWAS run. Quantile-quantile (QQ) plots of the genome-wide marginal p-values against the expected distribution of p-values can identify unknown population structure in the data leading to spurious results, as well as evidence of polygenic trait architecture. Variant quality control can include filtering variants with a high no-call rate, allele frequencies substantially out of Hardy-Weinberg equilibrium, imputed variants with poor imputation quality, and variants with very low allele frequencies.

d) Enumerate the Associated Loci, Generate Locus-Specific Association Plots and Cross-Reference with Published Loci

High-quality genome-wide significant loci can be further examined by visualizing the distribution of p-values of variants in the nearby genomic context, by using a visualization tool like LocusZoom, a suite of tools to provide fast visualization of GWAS results for research and publication, available for download at locuszoom.org. See R. J. Pruim et al., LocusZoom: regional visualization of genome-wide association scan results Bioinformatics 15; 26(18) pp. 2336-7 (September 2010). An absence of LD-linked variants at similar p-values for enrichment are often indicative of low quality or spurious associations. Another way to gain confidence in the GWAS results is to cross-reference the reported associations with existing, known variants associated with the trait of interest. It is expected that some or many of the known associated variants should be replicated in a new GWAS from the same population, with similar estimated effect sizes of the variants.

e) Perform Meta-Analysis with Existing Published GWAS

To increase power and identify significant variants that do not meet genome-wide significance in any single study, meta-analysis of association statistics across two or more studies can be performed. See the open source tool known as METAL for an example, described in the article of Cristen Willer et al., METAL: fast and efficient meta-analysis of genomewide association scans, Bioinformatics Application note Vol. 26 no. 17, pp. 2190-2191 (2010).

f) Repeat GWAS Step 210 and Conditional Association Discovery

When we use a model 110 that produces phenotype labels that are probabilities (not binary values) repeating the GWAS allows both conditional association discovery (e.g. genetic associations with a first phenotype, e.g., POAG that are not acting through changes to VCDR, a second phenotype) and potentially allowing novel associations to subclinical phenotypes. Conditional associations can identify genes or pathways not previously implicated in the disease etiology and thus shed light on novel biological mechanisms of the disease. For diseases which manifest as gradual changes to eye morphology, disease status predictions far from the {0, 1} classification states may represent subclinical phenotypes. GWAS on these continuous predictions boost statistical power and can identify novel associations.

### Other Examples

Alternative embodiments of this disclosure are contemplated, including extending the prediction capacity for other phenotypes from color fundus images. It is specifically contemplated that we can apply the procedures of FIGS. 1A and 1B to research in not just glaucoma genetics, but rather we can extend this work to diabetic retinopathy and age-related macular degeneration genetics.

Additionally, alternative data modalities can be used for the training dataset 102 and the cohort of interest 202 that are also routine clinical measurements including but not limited to electronic health records, medical imaging data, and laboratory values.

The features of this disclosure provides multiple benefits over existing phenotyping solutions.

First, the mechanism for phenotyping of FIG. 1A has a cost that is fixed as a function of the phenotype: the cost to label a dataset (step 104) from which to train the model 110 and then perform the model training. The marginal cost to phenotype an individual given this model is negligible. This contrasts with existing phenotyping mechanisms whose costs are dependent on the number of individuals in the target cohort of interest, and explained above the cost and effort to produce phenotype labels in such cohorts can be prohibitive.

Second, the application of this phenotyping method is not subject to individual biases as seen in self-reported data.

Third, this phenotyping method implemented in FIG. 1B can be used to retrospectively phenotype a cohort without requiring additional interaction with the individuals in the cohort, for example where the individuals cannot be found, or may have died.

Fourth, this phenotyping method produces more nuanced phenotypes than a binary label provides, allowing both conditional association discovery (e.g. genetic associations with POAG that are not acting through changes to VCDR) and potentially allowing novel associations to subclinical phenotypes.

We claim:

1. A method comprising:
obtaining a training dataset that includes a first plurality of records for a first plurality of individuals, wherein each record of the training dataset includes, for a respective individual, a phenotype status for the respective individual and clinical data of a specified type for the respective individual;
using the training dataset to train a machine learning model to generate a predicted phenotype status based on input clinical data;
obtaining a target dataset that includes a second plurality of records for a second plurality of individuals, wherein each record of the target dataset includes, for a respective individual, genomic data for the respective individual and clinical data of the specified type for the respective individual;
applying the machine learning model to the clinical data of the target dataset to generate, for each individual in the second plurality of individuals, a predicted target phenotype status; and
based on the genomic data of the target dataset and the predicted target phenotype statuses, determining, for the second plurality of individuals, at least one association between the genomic information and a first phenotype.

2. The method of claim 1, wherein the first phenotype is associated with glaucoma and wherein the specified type of clinical data comprises retinal fundus photographic images.

3. The method of claim 2, wherein the first phenotype comprises risk of glaucomatous optic neuropathy.

4. The method of claim 1, wherein determining, for the second plurality of individuals, at least one association between the genomic information and individual phenotype comprises performing a genome-wide association study (GWAS).

5. The method of claim 1, wherein the machine learning model comprises an ensemble of deep convolutional neural networks.

6. The method of claim 1, wherein the predicted target phenotype status comprises a continuous variable probability prediction.

7. The method of claim 1, further comprising:
based on the genomic data of the target dataset and the predicted target phenotype statuses, determining, for the second plurality of individuals, at least one association between the genomic information and a second phenotype, wherein the first phenotype is not associated with the second phenotype.

8. The method of claim 1, wherein the clinical data of the first plurality of records comprises electronic health records.

9. The method of claim 1, wherein the specified type of clinical data comprises medical imaging data.

10. The method of claim 1, wherein the specified type of clinical data comprises laboratory test values.

11. The method of claim 1, wherein determining at least one association between the genomic information and the first phenotype comprises identifying a set of one or more genomic loci.

12. An article of manufacture including a non-transitory computer-readable medium, having stored thereon program instructions that, upon execution by a computing device, cause the computing device to operations comprising:
obtaining a training dataset that includes a first plurality of records for a first plurality of individuals, wherein each record of the training dataset includes, for a respective individual, a phenotype status for the respective individual and clinical data of a specified type for the respective individual;
using the training dataset to train a machine learning model to generate a predicted phenotype status based on input clinical data;
obtaining a target dataset that includes a second plurality of records for a second plurality of individuals, wherein each record of the target dataset includes, for a respective individual, genomic data for the respective individual and clinical data of the specified type for the respective individual;
applying the machine learning model to the clinical data of the target dataset to generate, for each individual in the second plurality of individuals, a predicted target phenotype status; and
based on the genomic data of the target dataset and the predicted target phenotype statuses, determining, for the second plurality of individuals, at least one association between the genomic information and a first phenotype.

13. The article of manufacture of claim 12, wherein the first phenotype is associated with glaucoma and wherein the specified type of clinical data comprises retinal fundus photographic images.

14. The article of manufacture of claim 13, wherein the first phenotype comprises risk of glaucomatous optic neuropathy.

15. The article of manufacture of claim 12, wherein determining, for the second plurality of individuals, at least one association between the genomic information and individual phenotype comprises performing a genome-wide association study (GWAS).

16. The article of manufacture of claim 12, wherein the machine learning model comprises an ensemble of deep convolutional neural networks.

17. The article of manufacture of claim 1, wherein the predicted target phenotype status comprises a continuous variable probability prediction.

18. The article of manufacture of claim 12, wherein the operations further comprise:
based on the genomic data of the target dataset and the predicted target phenotype statuses, determining, for the second plurality of individuals, at least one association between the genomic information and a second phenotype, wherein the first phenotype is not associated with the second phenotype.

19. The article of manufacture of claim 12, wherein the clinical data of the first plurality of records comprises electronic health records.

20. The article of manufacture of claim 12, wherein the specified type of clinical data comprises medical imaging data.
